(19) United States
(12) Patent Application Publication
(10) Pub. No.: US 2024/0116881 A1
(43) Pub. Date: Apr. 11, 2024

(54) PROCESS FOR PREPARATION OF IPCONAZOLE & ITS INTERMEDIATES

(71) Applicants: UPL Mauritius Limited, Port Louis (MU); UPL Europe LTD, Warrington (GB)

(72) Inventors: Sebastian Ebner, Linz (AT); Johann Hiebl, Linz (AT); Michael König, Linz (AT); Samuel Ruehs, Linz (AT); Raffael Schuecker, Linz (AT); Cameron Gibb, Durham, NC (US)

(21) Appl. No.: 18/466,489

(22) Filed: Sep. 13, 2023

Related U.S. Application Data
(60) Provisional application No. 63/406,861, filed on Sep. 15, 2022.

(51) Int. Cl. C07D 249/08; B01J 21/18; B01J 23/44; B01J 23/755; C07C 45/62; C07C 45/69; C07C 45/73; C07C 45/81

(52) U.S. Cl. CPC C07D 249/08; B01J 21/18; B01J 23/44; B01J 23/755; C07C 45/62; C07C 45/69; C07C 45/73; C07C 45/81

(57) ABSTRACT

Processes for preparation of ipconazole, processes for preparation of a compound of formula (V) and processes for preparation of a compound of formula (VI), which is a key intermediate useful for preparation of ipconazole are disclosed.

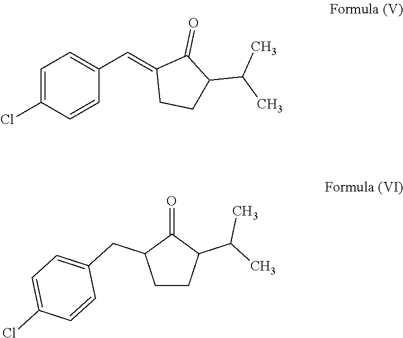 [SMILES: CC(C)C1CC/C(=C\c2ccc(Cl)cc2)C1=O ; CC(C)C1CCC(Cc2ccc(Cl)cc2)C1=O]

### CROSS REFERENCE TO RELATED APPLICATIONS

The present application claims the benefit of U.S. Provisional Application No. 63/406,861 filed on Sep. 15, 2022, incorporated herein by reference in its entirety.

### BACKGROUND

The present invention relates to a process for preparation of Ipconazole and its intermediates.

Ipconazole is a triazole fungicide that is widely used for seed treatment on various crops, turfgrass, ornamental flowers and conifers. It is a systemic, broad-spectrum fungicide seed dressing used to protect plants from soil borne and seed borne disease. Ipconazole is structurally similar to many other triazole compounds used as pesticides and is represented by formula (I) as follows.

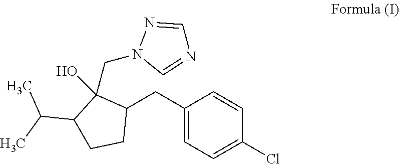 [SMILES: CC(C)C1CCC(Cc2ccc(Cl)cc2)C1(O)Cn1cncn1]

It is chemically known as 2-[(4-chlorophenyl)methyl]-5-(1-methylethyl)-1-(1H-1,2,4-triazol-1-ylmethyl)cyclopentanol.

EP0267778 discloses a process for preparation of azole derivative of formula (Ia). According to the process disclosed in said patent, an ester derivative of cyclopentane carboxylic acid of formula (a) is subjected to hydrolytic decarboxylation to obtain a cyclopentanone derivative of formula (b) which is then subjected to oxirane reaction to obtain oxirane of formula (c) which on reaction with 1,2,4-triazole provides desired azole derivative of formula (Ia). This reaction is a multistep reaction and represented as follows in Scheme I. However, this process is a complicated process.

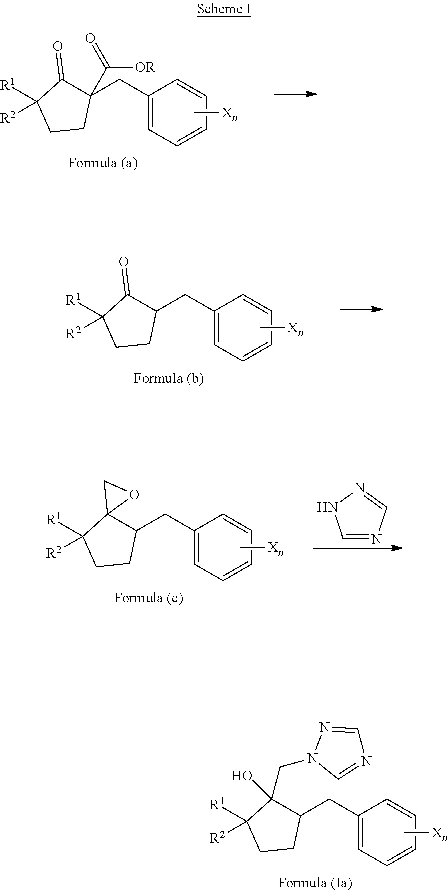

wherein R¹ and R² each independently represents a (C₁-C₅) alkyl group or a hydrogen atom; X represents a halogen atom; n represents 0, 1 or 2; and R represents a (C1-C5) alkyl group.

EP0329397 discloses a process for producing an azolylmethylcyclopentanol derivative by reacting 2-(4-chlorobenzyl)-5-(2-propyl)cyclopentanone with 1,2, 4-triazole and sulfoxonium methylide in a polar solvent or mixture of polar solvent and an alcohol in presence of a base. The reaction is represented in below Scheme II.

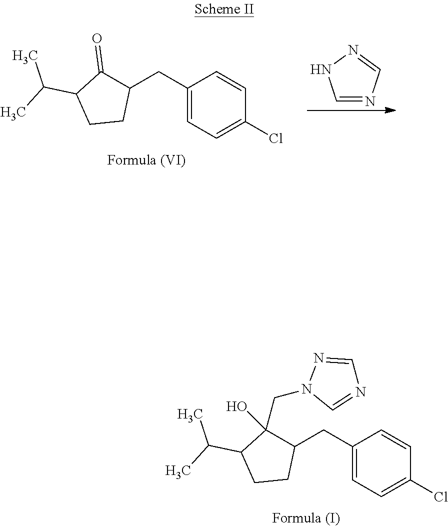 [SMILES: CC(C)C1CCC(Cc2ccc(Cl)cc2)C1=O ; c1nc[nH]n1 ; CC(C)C1CCC(Cc2ccc(Cl)cc2)C1(O)Cn1cncn1]

U.S. Pat. No. 5,466,816 discloses a process for preparing a derivative of azolylmethylcycloalkanol. This patent discloses a process for preparation of ipconazole by reacting a 2-(4-chlorobenzyl)-5-(2-propyl)cyclopentanone with 1,2, 4-triazole and a sulfonium compound in presence of metal oxide and an organic solvent. However, after the completion of reaction, cooling and filtration is required to remove large amount of solid by-product generated during the reaction. Hence, it can be concluded that this process is tedious as the solid waste generated during the process is high and the process is not eco-friendly.

To meet the requirement of obtaining high purity of ipconazole, all the above process requires purification of Ipconazole using column chromatography which is less desirable for the large-scale production.

Thus, there is still need for a process for preparation of Ipconazole and its intermediates which provides Ipconazole with high purity, and also overcomes the disadvantages of the processes described in the prior art.

### BRIEF SUMMARY

According to an aspect of the present invention, there is provided a process for preparation of a compound of formula (V) using cyclopentanone of formula (II).

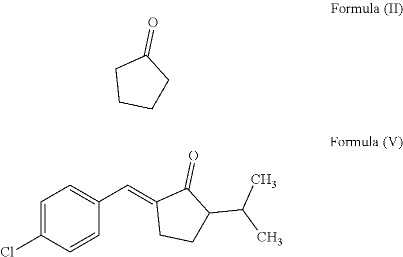 [SMILES: O=C1CCCC1 ; CC(C)C1CC/C(=C\c2ccc(Cl)cc2)C1=O]

According to another aspect of the present invention, there is provided a process for preparation of a compound of formula (VI), a key intermediate useful for preparation of Ipconazole.

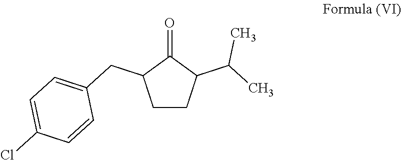 [SMILES: CC(C)C1CCC(Cc2ccc(Cl)cc2)C1=O]

comprising hydrogenating a compound of formula (V);
wherein the compound of formula (V) is prepared using a cyclopentanone of formula (II).

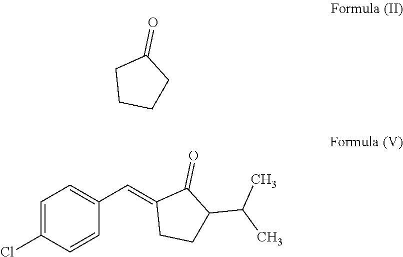 [SMILES: O=C1CCCC1 ; CC(C)C1CC/C(=C\c2ccc(Cl)cc2)C1=O]

According to an aspect of the present invention, there is provided a process for preparation of a compound of formula (VI)

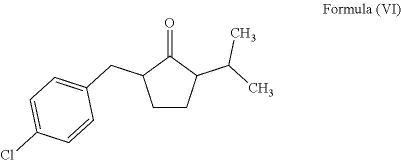 [SMILES: CC(C)C1CCC(Cc2ccc(Cl)cc2)C1=O]

comprising
a) reacting a cyclopentanone of formula (II) with acetone to obtain a compound of formula (III);

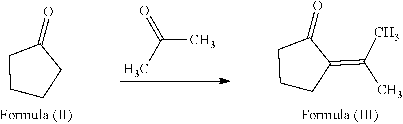

b) hydrogenating the compound of formula (III) to obtain a compound of formula (IV);

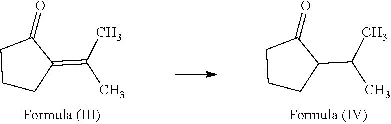

c) reacting the compound of formula (IV) with 4-chlorobenzaldehyde to obtain a compound of formula (V) and

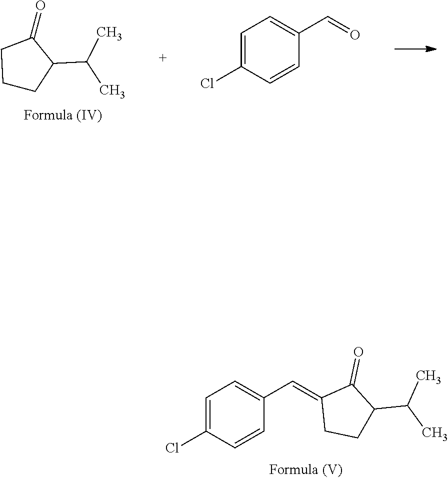

d) hydrogenating the compound of formula (V) to obtain a compound of formula (VI).

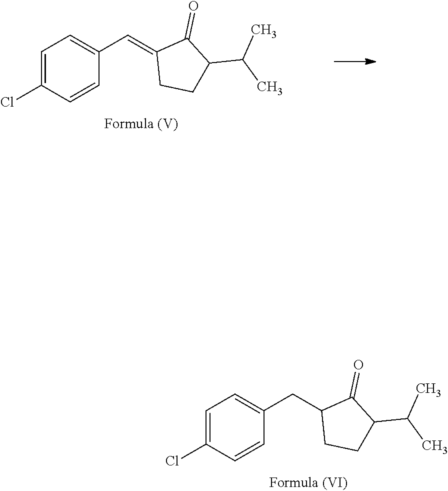 [SMILES: CC(C)C1CC/C(=C\c2ccc(Cl)cc2)C1=O ; CC(C)C1CCC(Cc2ccc(Cl)cc2)C1=O]

According to yet another aspect of present invention, there is provided a process for preparation of Ipconazole of formula (I)

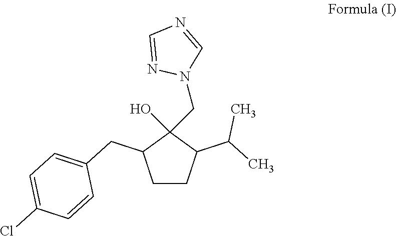 [SMILES: CC(C)C1CCC(Cc2ccc(Cl)cc2)C1(O)Cn1cncn1]

comprising hydrogenating a compound of formula (V);
wherein the compound of formula (V) is prepared by using a cyclopentanone of formula (II).

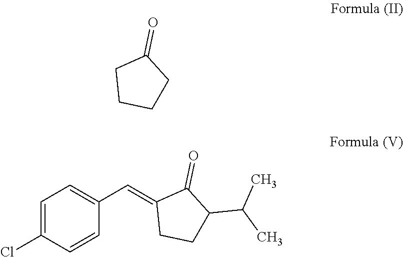 [SMILES: O=C1CCCC1 ; CC(C)C1CC/C(=C\c2ccc(Cl)cc2)C1=O]

### DETAILED DESCRIPTION

Those skilled in art will be aware that invention described herein is subject to variations and modifications other than those specifically described. It is to be understood that the invention described herein includes all such variations and modifications. The invention also includes all such steps, features, compositions and methods referred to or indicated in this specification, individually or collectively, and any and all combinations of any two or more said steps or features.

### Definitions

For convenience, before further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

The term “room temperature” unless stated otherwise, essentially means temperature in range of 20-35° C.

The term “purity” means purity as determined by HPLC (“High Pressure Liquid Chromatography”).

The term “about” or “approximately” as used herein is inclusive of the stated value and means within an acceptable range of deviation for particular value as determined by one of ordinary skill in the art, considering the measurement in question and the error associated with measurement of the particular quantity (i.e., the limitations of the measurement system). For example, “about” can mean within one or more standard deviations, or within ±10 or ±5 of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. It is understood that where a parameter range is provided, all integers within that range, and tenths thereof, are also provided. For example, “0.1-80%” includes 0.1%, 0.2%, 0.3%, etc. up to 80%. As used herein, the terms “comprising” “including,” “having,” “containing,” “involving,” and the like are to be understood to be open-ended, i.e., to mean including but not limited to.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

The term “crude Ipconazole” used herein refers to Ipconazole having low HPLC purity or obtained by the methods known to person skilled in art, having low HPLC purity.

The inventors of present invention have evolved a synthetic route for the preparation of Ipconazole and its intermediates which starts from easily available materials and employs mild reaction conditions and has simpler after-treatment procedures, thus making it suitable for large-scale production.

According to an aspect of the present invention, there is provided a process for preparation of a compound of formula (V) using cyclopentanone of formula (II).

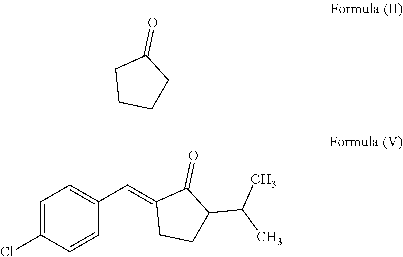 [SMILES: O=C1CCCC1 ; CC(C)C1CC/C(=C\c2ccc(Cl)cc2)C1=O]

In an embodiment, there is provided a process for preparation of a compound of formula (V)

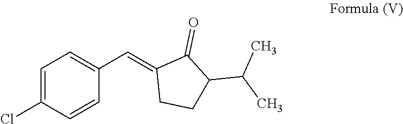 [SMILES: CC(C)C1CC/C(=C\c2ccc(Cl)cc2)C1=O]

wherein the process proceeds via cyclopentanone.

According to another aspect of the present invention, there is provided a process for preparation of a compound of formula (VI)

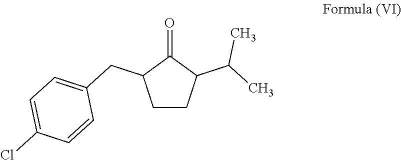 [SMILES: CC(C)C1CCC(Cc2ccc(Cl)cc2)C1=O]

comprising hydrogenating a compound of formula (V);
wherein compound of formula (V) is prepared using a cyclopentanone of formula (II).

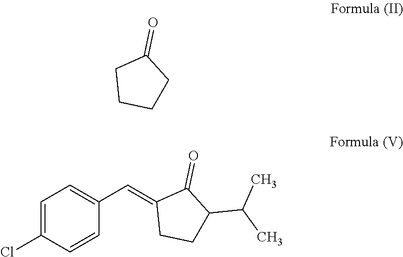 [SMILES: O=C1CCCC1 ; CC(C)C1CC/C(=C\c2ccc(Cl)cc2)C1=O]

According to an aspect of the present invention, there is provided a process for preparation of a compound of formula (VI)

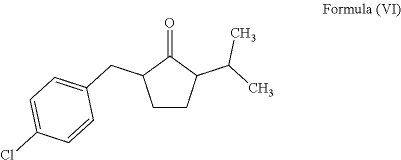 [SMILES: CC(C)C1CCC(Cc2ccc(Cl)cc2)C1=O]

comprising
a) reacting a cyclopentanone of formula (II) with acetone to obtain a compound of formula (III);

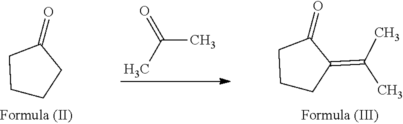

b) hydrogenating the compound of formula (III) to obtain a compound of formula (IV);

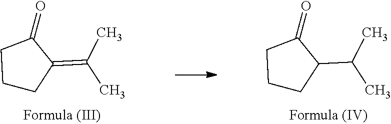

c) reacting the compound of formula (IV) with 4-chlorobenzaldehyde to obtain a compound of formula (V) and

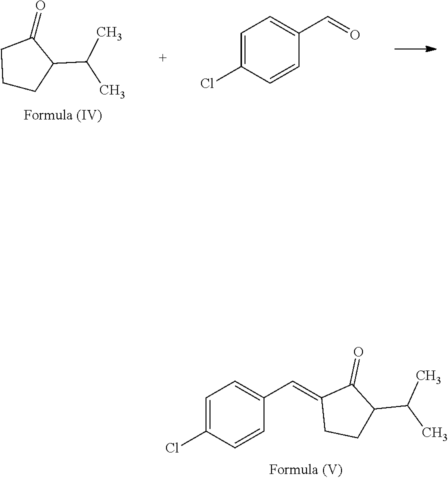

d) hydrogenating the compound of formula (V) to obtain a compound of formula (VI).

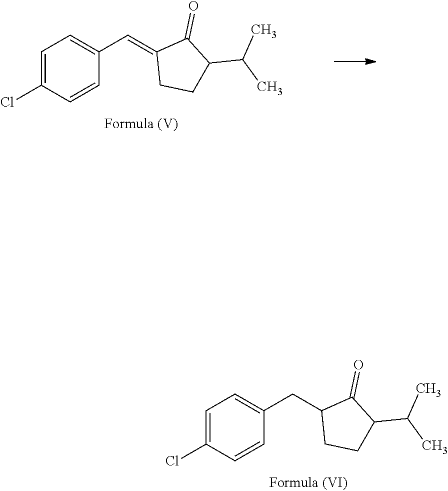 [SMILES: CC(C)C1CC/C(=C\c2ccc(Cl)cc2)C1=O ; CC(C)C1CCC(Cc2ccc(Cl)cc2)C1=O]

According to an embodiment, the step a) of the process is carried out in presence of a base.

In an embodiment, the base used is selected from an organic or an inorganic base.

In an embodiment, the organic base used is alkyl compounds of alkali metal. In an embodiment the alkyl compounds of alkali metal is selected from n-butyllithium; and triethylamine, pyridine, and the likes.

In an embodiment, the inorganic base used is selected from, but not limited to, alkaline metal alkoxide such as sodium methoxide, sodium ethoxide, potassium t-butoxide and the like; or an alkaline metal hydroxide such as sodium hydroxide, potassium hydroxide and the like.

In an embodiment, the amount of base used is in the range of 0.25 to 1 mole with respect to cyclopentanone of formula (II).

In an embodiment, the amount of acetone used is in the range of 1 to 15 moles, preferably 7 to 12 moles with respect to cyclopentanone of formula (II).

In an embodiment, acetone is added in a single lot or in multiple batches, preferably acetone is added in multiple batches.

According to an embodiment, the step a) of the process is carried out in presence of a suitable solvent.

The solvent used is selected from, but not limited to, water, alcohols like methanol, ethanol, isopropanol, n-propanol, butanol, tert-butanol, for example water is used as solvent.

In an embodiment the step a) of the process is carried out at temperature in the range from about 20 to 100° C. In an embodiment the step a) of the process is carried out at temperature in the range from about 20 to 70° C.

In preferred embodiment, the step a) of the process is carried out at temperature ranging from 20 to 35° C.

In an embodiment, after completion of reaction of the step a) of the process the reaction mixture is quenched with an acid and the pH is adjusted between 4 to 7.

In an embodiment, the acid used for quenching of the reaction, may be selected from an organic acid such as glacial acetic acid and the like; or an inorganic acid such as hydrochloric acid, nitric acid, sulfuric acid and the like.

In an embodiment, after completion of reaction of step a) of the process the reaction mixture is quenched with glacial acetic acid and the pH is adjusted between 4 to 7.

In an embodiment, the compound of formula (III) obtained has purity of more than 95%.

In an embodiment of the present invention, the step b) of the process is carried out in presence of a metal catalyst.

In an embodiment, the metal catalyst used is selected from, but not limited to, palladium, palladium on carbon, platinum, Raney-nickel and the like.

In an embodiment, the metal catalyst used is optionally supported on inert support such as carbon, alumina or silica.

In an embodiment, the step b) of the process is carried out in absence of the solvent.

In an embodiment, the step b) of the process is carried out in presence of an inert solvent.

In an embodiment, the inert solvent is selected from, but not limited to, lower alkanols such as methanol, ethanol, isopropanol and n-butanol; ethers, such as tetrahydrofuran, ethyl ether, 1,2-dimethoxyethane and diethylene glycol dimethylether; esters such as ethyl acetate, methyl propionate and dimethylsuccinate; and dimethylformamide. Particularly preferred such solvents are methanol and ethanol.

In an embodiment, the step b) of the process is carried out at temperature in the range from about 10 to 100° C., and pressure ranging from about 1-10 atmospheres.

In an embodiment, the step b) of the process is carried out at temperature in the range from about 20 to 100° C. In an embodiment the step b) of the process is carried out at temperature in the range from about 20 to 70° C.

In an embodiment, the compound of formula (III) obtained in the step b) having purity of more than 95%.

In an embodiment, the step c) of the process is carried out in presence of a base selected from, an organic or inorganic base.

In an embodiment, the organic base used is selected from, but not limited to, alkyl compounds of alkali metal such as n-butyllithium; and triethylamine, pyridine, and the likes.

In an embodiment, the inorganic base used is selected from, but not limited to, alkali and alkaline earth metal hydroxides, such as lithium hydroxide, sodium hydroxide, potassium hydroxide, caesium hydroxide, magnesium hydroxide. calcium hydroxide, strontium hydroxide and barium hydroxide, and alkali metal and alkaline earth metal carbonates, such as sodium carbonate, potassium carbonate, calcium carbonate and magnesium carbonate, and oxides of the said elements which form hydroxides or carbonates.

In an embodiment the inorganic base is alkali metal hydroxide. In an embodiment, the alkali metal hydroxide is selected from sodium hydroxide or potassium hydroxide.

In an embodiment, the amount of base used in the step c) is in the range of 0.01 to 1 moles with respect to compound of formula (IV).

In an embodiment the amount of 4-chlorobenzaldehyde used in step c) is in the range of 0.5 to 2 moles with respect to compound of formula (IV).

In an embodiment, the step c) of the process is carried out in presence of a suitable solvent such as water, lower alcohol such as methanol ethanol, n-propanol, isopropanol; sulphones such as dimethyl sulphoxide; ethers such as tetrahydrofuran; amides such as dimethylformamide, N-methylpyrrollidone; or mixture thereof.

In an embodiment, the step c) of the process is carried out in presence of water as solvent.

The amount of solvent used in step c) is about 1 to 5 times with respect to the compound of formula (IV).

In an embodiment, the step c) of the process is carried out at temperature ranging from 20 to 180° C., at pressure of about 5 to 15 bar.

In another embodiment, the step c) further comprises a step of purifying the compound of formula (V).

In another embodiment, the process for purifying the compound of formula (V) comprises treatment of the compound of formula (V) with an alcohol.

In an embodiment the alcohol used is selected from C1 to C8 alcohols like methanol, ethanol, propanol, isopropanol, n-butanol, tert-butanol and the like.

Preferably, methanol is used for purification of compound of formula (V).

In another embodiment, the process for purifying the compound of formula (V) comprises one or more processes selected from,
i) making slurry of the compound of formula (V) with an alcohol and isolating pure compound of formula (V); or
ii) washing the compound of formula (V) with an alcohol to obtain pure compound; or
iii) dissolving the compound of formula (V) in the alcohol to obtain a solution and recrystallizing the compound of formula (V) from the solution obtained to obtain pure compound.

In an embodiment, the process for purifying the compound of formula (V) is carried out at temperature in the range from 0° C. to 100° C.

In an embodiment the compound of formula (V) obtained in the step c) is having purity of more than 95%.

In an embodiment of the present invention, the step d) of the process is carried out in presence of a metal catalyst.

In an embodiment the metal catalyst used is selected from, but not limited to, palladium, palladium on carbon, platinum, Raney-nickel and the like. Preferably, Raney-nickel is used.

In an embodiment the metal catalyst used is optionally supported on inert support such as carbon, alumina or silica.

According to an embodiment, the step d) of the process is carried out in presence of inert solvent selected from lower alkanols; ethers; esters and amides.

According to an embodiment, the step d) of the process is carried out in presence of inert solvent selected from, but not limited to, lower alkanols such as methanol, ethanol, isopropanol and n-butanol; ethers, such as tetrahydrofuran, ethyl ether, 1,2-dimethoxyethane and diethylene glycol dimethylether; esters such as ethyl acetate, methyl propionate and dimethylsuccinate; and dimethylformamide. Preferably, tetrahydrofuran is used.

In an embodiment, the step d) of the process is carried out at temperature range from about 0 to 50° C. Preferably, at temperature in the range from 0 to 20° C.

In an embodiment, the compound of formula (VI) obtained in the step d) having purity of at least 95%.

In a preferred embodiment, the present invention provides a process for preparation of a compound of formula (VI)

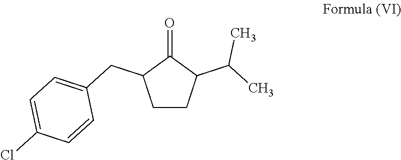 [SMILES: CC(C)C1CCC(Cc2ccc(Cl)cc2)C1=O]

comprising
a) reacting a cyclopentanone of formula (II) with acetone to obtain a compound of formula (III);

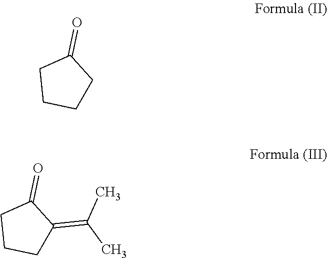

b) hydrogenating the compound of formula (III) to obtain a compound of formula (IV);

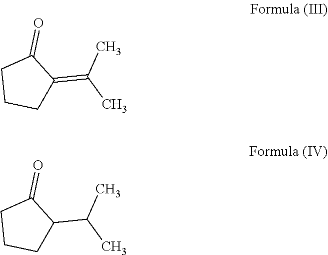

c) reacting the compound of formula (IV) with 4-chlorobenzaldehyde to obtain a compound of formula (V);

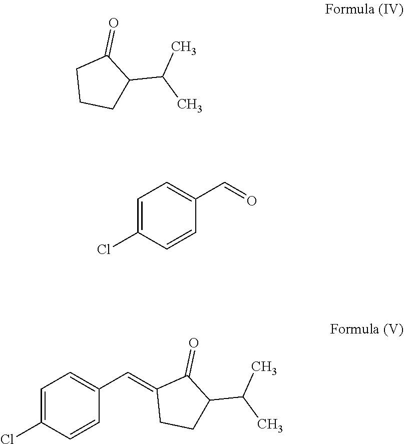

d) hydrogenating the compound of formula (V) to obtain a compound of formula (VI).

In yet another embodiment, there is provided a process for preparation of Ipconazole of formula (I)

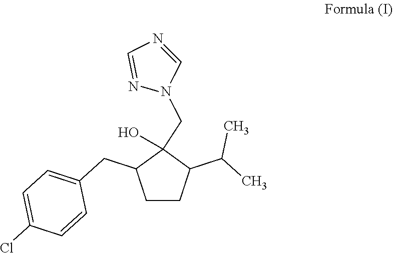 [SMILES: CC(C)C1CCC(Cc2ccc(Cl)cc2)C1(O)Cn1cncn1]

comprising hydrogenating a compound of formula (V);
wherein compound of formula (V) is prepared by using cyclopentanone of formula (II).

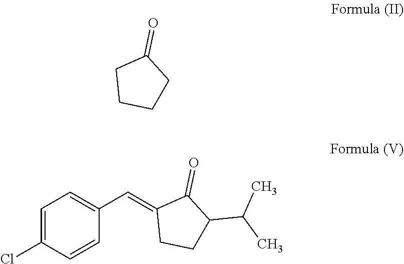 [SMILES: O=C1CCCC1 ; CC(C)C1CC/C(=C\c2ccc(Cl)cc2)C1=O]

According to an embodiment, there is provided a process for preparation of Ipconazole of formula (I)

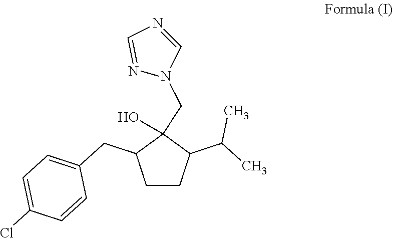 [SMILES: CC(C)C1CCC(Cc2ccc(Cl)cc2)C1(O)Cn1cncn1]

comprising
a) reacting cyclopentanone of formula (II) with acetone to obtain a compound of formula (III);

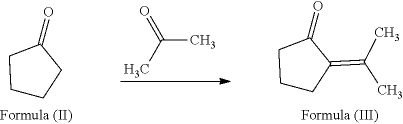

b) hydrogenating the compound of formula (III) to obtain a compound of formula (IV);

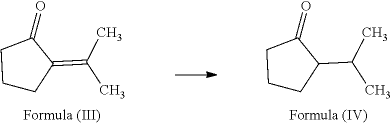

c) reacting the compound of formula (IV) with 4-chlorobenzaldehyde to obtain a compound of formula (V);

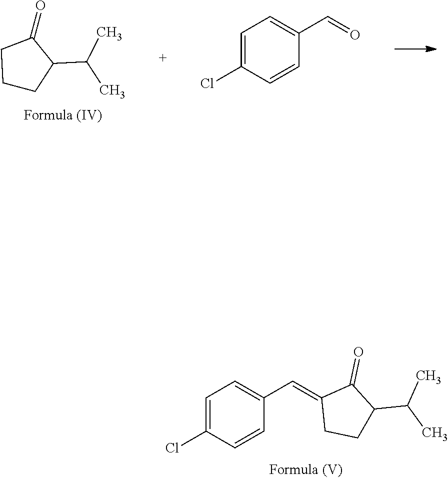

d) hydrogenating the compound of formula (V) to obtain a compound of formula (VI) and

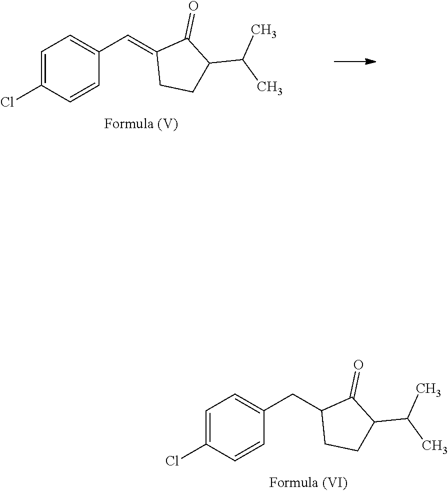 [SMILES: CC(C)C1CC/C(=C\c2ccc(Cl)cc2)C1=O ; CC(C)C1CCC(Cc2ccc(Cl)cc2)C1=O]

e) reacting the compound of formula (VI) with 1,2,4-triazole or its salt to obtain ipconazole of formula (I).

In an embodiment, the step e) reaction of the compound of formula (VI) with 1,2,4-triazole or its salt is carried out in presence of a base and a sulfonium halide or sulfoxonium halide; and a base.

In an embodiment the 1,2,4-triazole or its salt used is preferably in form of alkali metal salt of 1,2,4-triazole such as sodium 1,2,4-triazole salt, potassium 1,2,4-triazole salt, lithium 1,2,4-triazole salt, and the like.

The amount of 1,2,4-triazole or its salt used is in the range of 0.5 to 3 moles with respect to compound of formula (VI).

Optionally, the sulfonium halide or sulfoxonium halide and the base are added to the reaction intermittently.

The number of the intermittent addition of the sulfonium halide or sulfoxonium halide and the base, is not limited as long as the number is sufficient to achieve a predetermined purpose. Preferably, the number of intermittent additions may vary from 3 to 15 times in order to complete the reaction.

In an embodiment the sulfoxonium halide used may be selected from, but not limited to, trimethylsulfonium halide such as trimethylsulfonium iodide, trimethylsulfonium bromide, trimethylsulfonium chloride, and the like.

In an embodiment the sulfoxonium halide used may be selected from, but not limited to, trimethylsulfoxonium halide such as trimethylsulfoxonium iodide, trimethylsulfoxonium bromide, trimethylsulfoxonium chloride, and the like.

The amount of the sulfonium halide or sulfoxonium halide used is in the range from 0.5 to 3 moles with respect to compound of formula (VI).

In an embodiment, the base used is selected from an organic or inorganic base.

In an embodiment the organic base used may be selected from, but not limited to, alkyl compounds of alkali metal such as n-butyllithium; and triethylamine, pyridine, and the likes.

In an embodiment the inorganic base used may be selected from, but not limited to, carbonates of alkali metal such as sodium carbonate and potassium carbonate; hydroxides of alkali metal such as sodium hydroxide and potassium hydroxide; alkoxides of alkali metal such as sodium methoxide, sodium ethoxide, sodium tert-butoxide, and potassium tert-butoxide; alkali metal hydrides such as sodium hydride and potassium hydride; alkyl compounds of alkali metal such as n-butyllithium; and triethylamine, pyridine, and the like.

In an embodiment the amount of base used is in the range of 0.5 to 3 moles with respect to compound of formula (VI).

In an embodiment the reaction of compound of formula (VI) with 1,2,4-triazole or its salt is carried out in presence of an organic solvent selected from, but not limited to, N-methyl-2-pyrrolidone, dimethylformamide, dimethylacetamide, dimethyl sulfoxide or its mixture with a C1 to C5 alcohol like methanol, ethanol, propanol, isopropanol, tert-butanol and the like.

In an embodiment of the present invention, the reaction of compound of formula (VI) with 1,2,4-triazole or its salt is carried out at temperature ranging from 50 to 200° C.

According to an embodiment of the present invention, the process comprises of the step of purifying Ipconazole of formula (I) by treatment with a suitable organic solvent.

The process of purification of Ipconazole comprises at least one of the processes selected from i) washing, ii) making slurry of the crude Ipconazole with the suitable organic solvent and isolating purified Ipconazole of formula (I) or recrystallizing the crude Ipconazole by suitable method. The suitable organic solvent used may be selected from, but not limited to, saturated cyclic or acyclic hydrocarbons such as hexane, heptane, cyclohexane, methylcyclohexane and the like; saturated cyclic alcohols such as cyclopentanol, cyclohexanol, cycloheptanol and the like; or mixtures thereof. Preferably, the crude Ipconazole of formula (I) is first crystallised from cyclohexane and then from mixture of cyclohexanol in cyclohexane. In an embodiment, there is provided a process for the preparation of Ipconazole, wherein the process proceeds via a compound of formula (V) prepared according to the present process.

In another aspect, there is provided a process for the preparation of Ipconazole wherein the process proceeds via an intermediate of formula (VI) prepared according to the present process.

The process for preparation of Ipconazole according to present invention can be is illustrated by below scheme which follows:

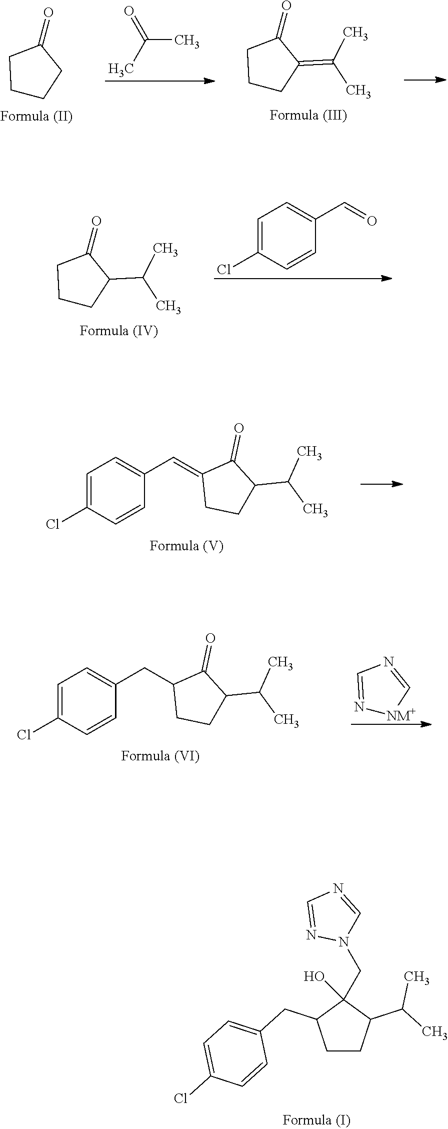

According to an embodiment of the present invention, the Ipconazole of formula (I) obtained by the process of present invention is having purity of at least than 95%.

### EXAMPLES

The following examples are presented to provide what is believed to be the most useful and readily understood description of procedures and conceptual aspects of this invention. The examples provided below are merely illustrative of the invention and are not intended to limit the same to disclosed embodiments. Variations and changes obvious to one skilled in the art are intended to be within the scope and nature of the invention.

### Example 1: Process for Preparation of Compound of Formula (III) [i.e. 2-Isopropylidenecyclopentanone]

A solution of 250 g (2.97 mol) cyclopentanone of formula (II) in 639 g (11 mol) acetone was added to a stirred cooled solution of aqueous sodium hydroxide solution (158 g of 30% NaOH solution in 2500 ml of water). The mixture was stirred for 3 hours at 20-25° C. and while maintaining the same temperature for another 8 hours, 990 ml of acetone was added in three batches of 330 ml each at interval of 2-3 hours. The reaction mixture was then quenched by adding 100 ml (1.51 mol) of glacial acetic acid and the pH was maintained between 5-6. The organic layer was separated, and the aqueous layer was extracted petroleum ether. The petroleum ether extract and organic layer were combined and washed with water and 5% sodium carbonate solution; solvent was removed on rotatory evaporator to obtain a residue. The residue thus obtained was distilled under vacuum to give 108 g pure titled compound of formula (III). Purity: 96%.

### Example 2: Preparation of Compound of Formula (IV) [i.e. 2-Isopropylcyclopentanone]

A Mixture of 2765 g (22.3 mol) compound of formula (III) obtained in Example 1 and 27.6 g (0.22 mol) of 5% w/w palladium-on-carbon catalyst was hydrogenated at 1.1 bar and 20-25° C. for 5 hours. After completion of reaction, the reaction mixture was passed over celite plug and the filtrate was distilled to afford 2325 g (83%) of titled compound 2-isopropylcyclopentanone [Purity: 99%].

### Example 3: Preparation of Compound of Formula (V) [i.e. 2-(4-Chlorobenzylidene)-5-Isopropyl-Cyclopentanone]

A solution of 60.1 g (0.92 mol) potassium hydroxide in 3250 ml deionized water was charged to a reactor and then 1300 g (10.2 mol) of compound of formula (IV) and 1460 g (10.2 mol) of 4-chlorobenzaldehyde as melt were added. The reactor was sealed, purged with nitrogen. The mixture was then heated to 120° C. for 6 hours. After completion of reaction, the mixture was cooled to 20-25° C. for 16 hours and the suspension was filtered over glass frit. The wet cake thus obtained was washed with 7.5 L of water and dried at 60° C. and 5 mbar on a rotary evaporator. The crude product thus obtained was recrystallised from methanol, filtered and dried to yield 2213 g of pure titled compound of formula (V). Purity: 99%.

### Example 4: Preparation of a Compound of Formula (VI) [i.e. 2-(4-Chlorobenzyl)-5-Isopropyl-Cyclopentanone]

A mixture of 1000 g (4.0 mol) of compound of formula (V) and 1500 ml of tetrahydrofuran was cooled to 8° C. and 200 g Raney-Nickel was added to it. The hydrogenation reaction was carried out at 8° C. to 12° C. under pressure until theoretical amount of hydrogen was absorbed.

Raney-Nickel catalyst was removed through filtration from the reaction mixture and the filtrate collected was evaporated to dryness under reduced pressure to obtain residue. The residue obtained was distilled under vacuum to give 769 g of titled compound of formula (VI). Purity: 99.5%.

### Example 5: Preparation of Ipconazole of Formula (I)

To 204 g (2.2 mol) of sodium 1,2,4-triazole salt was added 424 g (1.7 mol) of compound of formula (VI), and 1110 g of N-methyl-2-pyrrolidone to obtain a reaction mixture. The reaction mixture was heated to 112° C. and then 203 g (2.1 mol) sodium tert-butoxide and 469 g (2.1 mol) of trimethylsulphoxonium iodide were dividedly added in 3.5 hours. After the addition was completed, the mixture was further heated for 1 hour at same temperature. After completion of reaction, the reaction mixture was cooled to 60° C. and was quenched with 3320 ml of water. The mixture was then extracted with ethyl acetate and organic layer was separated. The organic layer thus obtained was washed with 3.5% sodium chloride solution and then concentrated to dryness under pressure to obtain crude ipconazole of formula (I). The crude product was then crystallized first from cyclohexane and then from mixture of 196 g 25% cyclohexanol in cyclohexane to obtain 160 g of ipconazole. Purity: 99.5%.

We claim:

1. A process for preparation of a compound of formula (V) 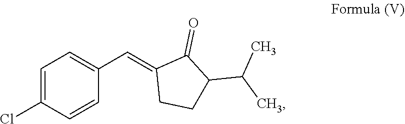 [SMILES: CC(C)C1CC/C(=C\c2ccc(Cl)cc2)C1=O]
wherein the process proceeds via cyclopentanone.

2. A process for preparation of a compound of formula (VI): 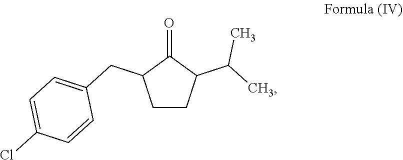 [SMILES: CC(C)C1CCC(Cc2ccc(Cl)cc2)C1=O]
the process comprising:
reacting a cyclopentanone of formula (II) with acetone to obtain a compound of formula (III); 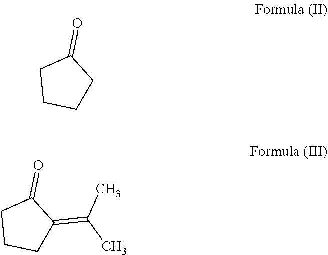
hydrogenating the compound of formula (III) to obtain a compound of formula (IV): 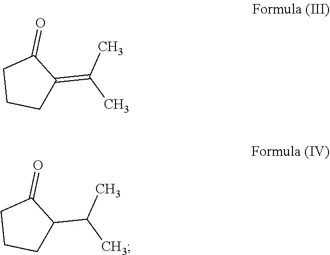
reacting the compound of formula (IV) with 4-chlorobenzaldehyde to obtain a compound of formula (V); 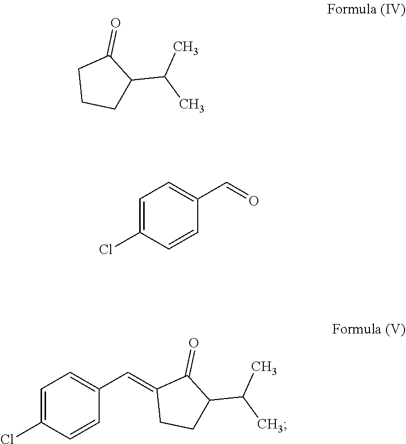
and
hydrogenating the compound of formula (V) to obtain the compound of formula (VI).

3. The process as claimed in claim 2, wherein hydrogenating the compound of formula (III) is carried out in absence of a solvent.

4. The process as claimed in claim 2, wherein reacting the cyclopentanone and hydrogenating the compound of formula (III) are carried out at a temperature from about 20 to 70° C.

5. The process as claimed in claim 2, wherein hydrogenating the compound of formula (III) and hydrogenating the compound of formula (V) are carried out in presence of a metal catalyst selected from a group consisting of palladium, palladium on carbon, platinum and Raney-nickel.

6. The process as claimed in claim 2, wherein reacting the compound of formula (IV) with 4-chlorobenzaldehyde to obtain the compound of formula (V) further comprises purifying the compound of formula (V) by treating the compound of formula (V) with an alcohol.

7. The process as claimed in claim 6, wherein the alcohol is selected from C₁ to C₈ alcohols, preferably methanol.

8. A process for preparation of ipconazole, wherein the process proceeds via an intermediate of formula (VI) prepared according to claim 2.

9. A process for preparation of Ipconazole of formula (I) 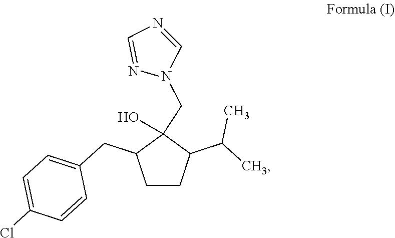 [SMILES: CC(C)C1CCC(Cc2ccc(Cl)cc2)C1(O)Cn1cncn1]
the process comprising:
reacting cyclopentanone of formula (II) with acetone to obtain a compound of formula (III); 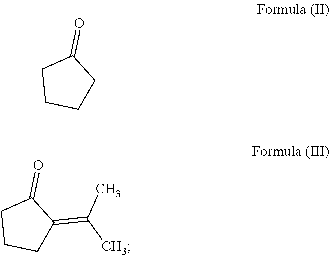
hydrogenating the compound of formula (III) to obtain a compound of formula (IV): 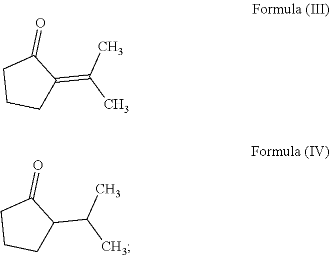
reacting the compound of formula (IV) with 4-chlorobenzaldehyde to obtain a compound of formula (V); 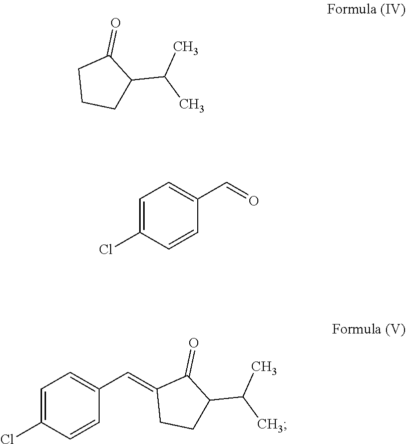
and
hydrogenating the compound of formula (V) to obtain a compound of formula (VI) and 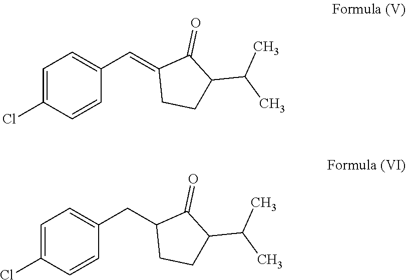 [SMILES: CC(C)C1CC/C(=C\c2ccc(Cl)cc2)C1=O ; CC(C)C1CCC(Cc2ccc(Cl)cc2)C1=O]
reacting the compound of formula (VI) with 1,2,4-triazole or its salt to obtain ipconazole of formula (I).
